(19) United States
(12) Patent Application Publication
(10) Pub. No.: US 2004/0063719 A1
(43) Pub. Date: Apr. 1, 2004

(54) Combination therapy using antihypertensive agents and endothelin antagonists

(75) Inventors: Michael A. Adams, Kingston (CA); Taben M. Hale, Kingston (CA); Jeremy P.W. Heaton, Gananoque (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Queen's University at Kingston, Kingston (CA); Callegy Pharmaceuticals, Inc., South San Francisco, CA

(21) Appl. No.: 10/429,197

(22) Filed: May 2, 2003

Related U.S. Application Data
(63) Continuation-in-part of application No. 10/192,281, filed on Jul. 9, 2002.
Application No. 10/192,281 is a continuation of application No. 09/902,787, filed on Jul. 12, 2001, now Pat. No. 6,458,797.
Application No. 09/902,787 is a continuation of application No. 09/382,749, filed on Aug. 25, 1999, now Pat. No. 6,284,763.
(60) Provisional application No. 60/098,178, filed on Aug. 26, 1998.
(60) Provisional application No. 60/377,917, filed on May 2, 2002.

(51) Int. Cl. A61K 31/496; A61K 31/4709; A61K 31/41; A61K 31/401

(52) U.S. Cl. 514/252.17; 514/423; 514/411; 514/381; 514/308

(57) ABSTRACT

The present invention provides a method for a more efficacious treatment of a vascular condition through the administration of a therapeutically effective amount of a combination of an anti-pressor agent, an endothelin antagonist, and a sex hormone for repetitive cycles of on/off-treatment. In one embodiment, the invention provides a method for the prevention of tolerance induced by an anti-pressor agent via the inclusion of an endothelin antagonist in a combination therapy approach to remodel vascular structure and treat vascular conditions associated with a male or female sexual dysfunction, atherosclerosis, renal failure, hypertension, congestive heart failure, diabetic nephropathy, and diabetic neuropathy. The anti-pressor agent comprises one or more compounds such as prostaglandin-E₁, an ACE inhibitor, an angiotensin-II receptor antagonist, an α₁-adrenergic receptor antagonist, a β-adrenergic receptor antagonist, a calcium channel blocker, an activator of guanylyl cyclase or adenyl cyclase, a phosphodiesterase inhibitor, and hydralazine. The endothelin antagonist comprises one or more compounds such as a peptidal endothelin antagonist, a non-peptidal endothelin antagonist, and an inhibitor of endothelin converting enzyme. Such a combination therapy approach enhances the efficacy of the anti-pressor agent and enables an increase in the frequency and duration of anti-pressor administrations for the long term treatment of vascular conditions.

### CROSS-REFERENCES TO RELATED APPLICATIONS

This application is a continuation-in-part of U.S. application Ser. No. 10/192,281, filed Jul. 9, 2002, which is a continuation of U.S. application Ser. No. 09/902,787, filed Jul. 12, 2001, now U.S. Pat. No. 6,458,797, which application is a continuation of U.S. application Ser. No. 09/382,749, filed Aug. 25, 1999, now U.S. Pat. No. 6,284,763, and which application claims priority to U.S. Provisional Application No. 60/098,178, filed Aug. 26, 1998. The present application claims priority to U.S. Provisional Application No. 60/377,917, filed May 2, 2002. All the foregoing applications are incorporated herein by reference in their entirety.

### FIELD OF THE INVENTION

The present invention relates to medical methods of treatment, pharmaceutical compositions, and use of anti-pressor agents, endothelial antagonists, and sex hormones to manufacture such pharmaceutical compositions. More particularly, the present invention is concerned with methods for providing more efficacious treatment regimens for the administration of agents which act in the long term management of sexual dysfunction in both males and females.

### BACKGROUND OF THE INVENTION

The physiology of an erection or sexual arousal in both the male and female involves central nervous system initiation, neural pathway activation, and vascular smooth muscle relaxation. This signaling mediates vasodilation of the penile, clitoral labial, and vaginal arterial blood vessels and the trabecular meshwork of smooth muscle. The resulting decrease in vascular resistance promotes an increase in arterial inflow and the filling of the corpora cavernosa in the penis and clitoris. Subsequent to there being an appropriate high rate of inflow, the cavernosal “filling” results in occlusion of the sub-tunical veins and full rigidity. The rate of inflow is critical because if there is not enough volume change, venous occlusion can not take place. A selective structurally-based increase in penile resistance produces a substantial impediment to inflow. That is, if penile or clitoral vascular structure, or the vascular structure immediately “up-stream” from the genitalia, is more constrained than the rest of the circulation, there would be a “mismatching” of perfusion pressure and selective resistance, i.e. genital arterial insufficiency. On the other hand, it is likely that when hypertension is first established and there is a generalized up-regulation of structurally-based vascular resistance in all vessels, there would not be any deleterious effect on erectile function because of a “matching” between perfusion pressure and resistance. That is, despite the hypertrophy of the penile vasculature, the arterial pressure is proportionally elevated thereby allowing for adequate blood flow to the penis.

Pathological changes in the genital vasculature and alterations in function control systems have been shown to have a deleterious impact on erectile dysfunction. Local factors such as endothelin and sympathetic nerve mediated release of catecholamines have been shown to be important players in detumescence, but they also are likely to increase trophic responses in this tissue. The physiology of penile and clitoral erection and the structural maintenance of the tissue depends upon a balance between control systems that involve endothelial cells, vascular smooth muscle cells, fibroblasts, extracellular matrix, and nerves. Any shift in the balance of these control systems to either towards trophic responses such as vascular hypertrophy, focal fibrosis, or generalized production of the extracellular matrix or to the extremes of functional control systems can result in erectile dysfunction. Further, as structure and function are so closely related, it is becoming increasingly important in understanding the mechanisms of erectile dysfunction that we investigate the reciprocal impact of structural changes on function and of changes in functional control systems on structure.

The clitoris is the homologue of the penis, arising from the embryological genital tubercle. As a result, the two organs have similar structural and arousal response mechanisms. The clitoris consists of a cylindrical, erectile organ composed of three parts: the outermost glans or head, the middle corpus or body, and the innermost crura. The body of the clitoris consists of paired corpora cavemosa of about 2.5 cm in length and lacks a corpus spongiosum. During sexual arousal, blood flow to the corpora cavemosa of the clitoris cause their enlargement and tumescence.

The clitoris plays a major role during sexual activity in that it contributes to local autonomic and somatic changes causing vaginal vasocongestion, engorgement, and subsequent effects, lubricating the introital canal making the sexual act easier, more comfortable, and more pleasurable.

Vaginal wall engorgement enables a process of plasma transduction to occur, allowing a flow through the epithelium and onto the vaginal surface. Plasma transduction results from the rising pressure in the vaginal capillary bed during the sexual arousal state. In addition, there is an increase in vaginal length and lumenal diameter, especially in the distal ⅔ of the vaginal canal.

It has been well established that the generation of penile and clitoral erections and vaginal and labial engorgement are greatly dependent on adequate blood flow to the vascular beds which feed these organs. Both smooth muscle relaxation of the corpora cavernosa as well as the vasodilation of genital arterial vessels mediate the physiological response. One of the major fundamental etiologies of erectile dysfunction is, thus, inadequate genital arterial inflow. If there is an inappropriate structural narrowing in the supporting vasculature that is not associated with an increase in perfusion pressure, the blood flow into the organs at maximum dilation may be reduced and therefore be insufficient for the generation of an erection. There is increasing recognition that erectile dysfunction, although associated with, may appear prior to the onset of clinical signs of cardiovascular disease and therefore may be an early harbinger of progressing changes.

In both the male and female human, the aorta bifurcates on the fourth lumbar vertebra into the common iliac arteries. The common iliac arteries pass laterally, behind the common iliac veins, to the pelvic brim. At the lower border of the fifth lumbar vertebra, the common iliac arteries divide into internal and external branches. The internal iliac artery supplies blood to all of the organs within the pelvis and send branches through the greater sciatic notch to supply the gluteal muscles and perineum. After passing over the pelvic brim, the internal iliac artery divides into anterior and posterior trunks.

The anterior trunk of the internal iliac artery branches into the superior vesical artery, the inferior vesical artery, the middle rectal artery, the uterine artery, the obturator artery, the internal pudendal artery, and the inferior gluteal artery. The internal pudendal artery supplies blood to the perineum. The artery passes out of the pelvis around the spine of the ischium and back on the inside surface of the ischeal tuberosity and inferior ramus to lie in the pudendal canal. The branches from the internal pudendal artery are the inferior rectal artery which supplies the anal sphincter, skin and lower rectum; the perineal artery which supplies the scrotum in the male and the labia in the female; the artery of the bulb which supplies erectile tissue, the deep dorsal arteries of the penis or deep artery of the clitoris.

It has been demonstrated in several forms of experimental hypertension that “slow pressor mechanisms” such as hypertrophic structural changes in the vasculature can almost completely account for the long-term resistance changes associated with the elevated arterial pressure. Based on Poiseuille's law, it has been shown that vascular resistance in an intact vascular bed is a function of the overall hemodynamic effect of all lumen radii, the number of blood vessels, the length of the vessels, and the blood viscosity. In hypertension, increased vascular resistance is most potently conferred by a structurally-based decrease in the radius of the lumen of arterioles and small arteries, and also potentially by arteriolar rarefaction, whereby even a small change in the average arteriolar radii throughout a vascular bed has a dramatic influence on the resistance to flow. Further, it has been demonstrated that such structural changes can precede the onset of hypertension and therefore may be an initiating mechanism.

Vascular beds in which there is chronic diminished blood flow suffer a degree of pathogenic vascular degradative modeling over time in response to static or circulatory hypoxia. That is, as a normal reaction to diminished blood flow, the lumen in these arteries diminishes in diameter over time, causing decreased blood flow and/or higher pressure during periods of peak blood flow. Those portions of the ilio-hypogastric-pudendal arterial bed which directly feed blood to the sex organs are examples of such less frequently used arterial beds. Because incidents of major blood inflow to the sexual organs are less frequent than to most other organs, a gradual hypoxic response is seen over time in the vasculature directly feeding these organs and in the organs themselves. The body has a self-regulating mechanism to combat this pathogenic modeling: it is known, for example, that in the human male there are a number of spontaneous nocturnal erections which occur as a result of the body's mechanism for combating hypoxia in penile tissue. Nevertheless, the arteries in a normal flaccid penis and the un-enlarged clitoris and labia are constricted. As a result, typical oxygen concentrations in such tissues are closer to venous rather than arterial oxygen levels. Periodic vasodilation of the penis and clitoris increases oxygen levels in these tissues. The higher oxygen levels supplied to tissue in the penis and clitoris, as well as vasodilation itself, shut down adverse metabolic processes such as TGF-β production and pathogenic vascular wall modeling, which result in long-term tissue damage.

Therefore, it is the differential changes in genital vascular resistance that is likely to be a critical issue in erectile function. That is, if such vascular structural changes take place in the genitalia in the absence of hypertension or systemic changes in vessel structure, there would not be the increase in arterial pressure required to compensate for the increased resistance. It may be that this condition could occur as an early indicator of progressing cardiovascular disease. The appearance of erectile dysfunction preceding the global clinical signs of hypertension may, in fact, suggest an increased susceptibility of this vascular bed to pathological changes.

In view of the foregoing, there is a need in the art for more efficacious treatment regimens for vascular conditions associated with a male or female sexual dysfunction, atherosclerosis, renal failure, hypertension, congestive heart failure, diabetic nephropathy, and diabetic neuropathy. For example, it is known that the development of tolerance to a drug-induced effect could reduce the overall efficacy of such a treatment. Thus, there is a need to address the side effects of tolerance in order to provide the most effective therapeutic strategy for the treatment of these vascular conditions. The present invention fulfills this and other needs.

### SUMMARY OF THE INVENTION

In one embodiment, the present invention provides a method for long term management of a vascular condition in males and females through a combination therapy approach using at least two agents selected from the group consisting of an anti-pressor agent, an endothelin antagonist, and a sex hormone. In certain aspects, the combination produces a persistent lowering of mean arterial pressure (MAP). Preferably, the MAP does not progressively decrease in magnitude with repetitive treatment cycles, enabling an increase in the frequency of anti-pressor administrations. In another embodiment, the present invention provides a method to treat a male or female sexual dysfunction, atherosclerosis, renal failure, hypertension, congestive heart failure, diabetic nephropathy, and diabetic neuropathy, wherein the method comprises administering to a patient in need of such treatment a therapeutically effective amount of a combination of at least two agents selected from the group consisting of an anti-pressor agent, an endothelin antagonist, and a sex hormone.

In a further embodiment, the present invention provides a method to treat a male or female sexual dysfunction, wherein the method comprises administering to a patient in need of such treatment a therapeutically effective amount of a combination of at least two agents selected from the group consisting of an anti-pressor agent, an endothelin antagonist, and a sex hormone. In yet a further embodiment, the present invention provides the use of a combination of at least two agents selected from the group consisting of an anti-pressor agent, an endothelin antagonist, and a sex hormone, for the manufacture of pharmaceutical compositions to treat a male or female sexual dysfunction, atherosclerosis, renal failure, hypertension, congestive heart failure, diabetes mellitus I and II and associated pathologies such as diabetic nephropathy, and diabetic neuropathy.

In certain aspects, a combination of at least two agents selected from the group consisting of an anti-pressor agent, an endothelin antagonist, and a sex hormone are co-administered for at least two treatment cycles separated by a drug-free period. The duration of a combination treatment and a drug-free period can vary for each cycle. In certain instances, the administration of an endothelin antagonist can increase the frequency or duration of administration of the anti-pressor agent, by, for example, reducing or eliminating tolerance for the anti-pressor agent. In certain instances, administration of an endothelin antagonist can increase the efficacy of the anti-pressor agent. In certain instances, the sex hormone is administered continuously throughout the treatment period. In one embodiment, the invention provides a method for the prevention of tolerance induced by an anti-pressor agent via the inclusion of an endothelin antagonist in a combination therapy approach to remodel vascular structure and treat vascular conditions associated with a male or female sexual dysfunction, atherosclerosis, renal failure, hypertension, congestive heart failure, diabetes mellitus I and II and associated pathologies such as diabetic nephropathy, and diabetic neuropathy.

These and other embodiments will become more apparent when read with the accompanying detailed description and figures which follow.

### BRIEF DESCRIPTION OF THE DRAWINGS

FIG. 1 is a representative cumulative α₁-adrenoreceptor concentration-response curve for administration of several doses of methoxamine (MXA) to a spontaneously hypertensive rat. Arrows indicate the point of drug delivery to the penile vascular bed at the concentrations labeled in the Figure. Each concentration of MXA was infused for a period of ten minutes, at which time a plateau was reached. The point marked “yield” in the Figure represents the pressure at maximum constriction of the blood vessels in the vascular bed. This maximum constriction was achieved by administration of a “cocktail” containing a mixture of vasopressin (20 g/mL), angiotensin-II (200 g/mL), and methoxamine (64 g/mL).

FIG. 2 shows the average α₁-adrenoreceptor concentration-response curves for administration of methoxamine (MXA) to both the penile vascular bed and hindlimb vascular bed perfusion preparations of the spontaneous hypertensive rat (SHR) and the normotensive Sprague-Dawley rat (SD). FIGS. 2a and 2b represent, respectively, the curves for administration to the penile vascular beds of the SHR and SD rat strains. FIGS. 2c and 2d represent, respectively, the curves for administration to the hindlimb vascular beds of the SHR and SD rat strains.

FIGS. 3a and 3b show the structurally-based vascular resistance asserted at (a) maximum dilation or (b) maximum constriction for the penile and hindlimb perfusion vascular preparations for the spontaneously hypertensive rat (SHR) and the normotensive Sprague-Dawley rat (SD).

FIG. 4 is a schematic representation depicting structural differences in blood vessels in the spontaneously hypertensive rat (SHR) and the normotensive Sprague-Dawley rat (SD) and the expected impact on resistance to blood flow.

FIGS. 5a and 5b show (a) mean arterial pressure (MAP) profiles of spontaneously hypertensive rats treated with three cycles of either enalapril or losartan, and control animals; and (b) a graph comparing the change in MAP of each group over the time corresponding to each cycle of treatment.

FIGS. 6a and 6b are graphs comparing treatment with enalapril and losartan on the daily rate of change of mean arterial pressure (MAP) upon (a) initiation or (b) cessation of therapy.

FIG. 7 is a graph comparing the overall persistent lowering of mean arterial pressure (MAP) versus control induced by different treatment protocols for enalapril and losartan.

FIG. 8 is a graph illustrating the effects of sodium manipulation on mean arterial pressure (MAP) in animals treated with three cycles of either enalapril and losartan and control animals.

FIGS. 9a and 9b are graphs comparing the perfusion pressures of animals treated with losartan for 14 days or controls at (a) maximum dilation or (b) maximum constriction.

FIG. 9c is a graph comparing the left ventricular mass of animals treated with losartan for 14 days or controls.

FIGS. 10a and 10b are graphs comparing the levels of endothelin present in control animals and animals on day 10 of the first and third cycles of treatment with enalapril in (a) the renal medulla and (b) the renal cortex.

### DETAILED DESCRIPTION OF THE INVENTION

The present invention provides the use of combinations of anti-pressor agents, endothelin antagonists, and sex hormones to remodel vasculature and treat vascular conditions associated with a male or female sexual dysfunction, atherosclerosis, renal failure, hypertension, congestive heart failure, diabetic nephropathy, and diabetic neuropathy. A method for the combined administration of at least two agents selected from an anti-pressor agent, an endothelin antagonist, and a sex hormone provided by the present invention enhances the efficacy of the anti-pressor agent and allows for an increase in the frequency of anti-pressor administrations for the long term management of vascular conditions.

There has been some controversy in the literature as to the correct definition of the term “vascular remodeling,” as evidenced by the exchange of letters in the Journal of Hypertension, 15: 333-337 (1997). The controversy in the nomenclature centers, in part, around the use of the terms “hypotrophic,” “eutrophic,” and “hypertrophic” as modifiers for the term “remodeling” as well as the use of the prefix “re-” in the word “remodeling.”

The “trophic” terms have been objected to because of their suggestion that some sort of growth change accompanies the observed vascular changes. The term “remodeling” was initially applied in the literature to the observation in spontaneously hypertensive rats and in hypertensive humans that the interior lumen radius (r₁) of blood vessels was greatly diminished while vessel wall mass (w) remained constant. The result was an observed increase in the ratio of w/r₁ which correlated with blood pressure elevation. The term “remodeling” was applied to the observed phenomenon, primarily because of the surprising consistency in total wall mass. It was thought that some sort of remodeling of the internal cellular structure of the blood vessel had occurred which permitted a change in lumen radius without a corresponding change in vessel wall mass.

Lacking a general consensus of the term “vascular remodeling” in the medical community, the term “pathogenic vascular degradative modeling” will be applied throughout this specification and the appended claims to denote the pathogenic or degradative increase in the ratio w/r₁ of vasculature, irrespective of the cause. The term “vascular remodeling,” as used throughout this specification and the appended claims, will mean the amelioration, inhibition, or reversal of pathogenic vascular degradative modeling; that is the amelioration, inhibition, or reversal of the increase in the ratio of vascular w/r₁.

“Sexual dysfunction” (SD) as used herein includes aspects of male dysfunction such as erectile dysfunction (ED), priapism, and premature ejaculation, and aspects of female dysfunction and urogenital aging such as decreased vaginal lubrication, decreased vaginal engorgement, pain during intercourse such as, for example, dyspareunia, an urogenital infection; and urogenitalia as affected by post-menopause, diabetes, vascular disease, an estrogen depletion condition, idiosyncratic vaginal dryness, vaginismus, vulvodynia (including vulvar vestibulitis), interstitial cystitis, nonspecific urethritis (i.e., nonspecific pain and/or burning of the urinary tract), and a variety of sexual dysfunctions including, but not limited to, female sexual arousal disorders, hypoactive desire disorders and female sexual orgasmic disorders.

The term “vascular condition” as used herein applies to a condition where regional circulation exhibits inappropriate vasoconstriction, lack of vasodilation, or diminished vasodilation, such as, for example, a male or female sexual dysfunction, atherosclerosis, renal failure, hypertension, congestive heart failure, diabetes mellitus I and II and associated pathologies such as diabetic nephropathy, and diabetic neuropathy.

The term “therapeutically effective amount of a combination of” refers to a combined amount of at least two agents selected from an anti-pressor agent, an endothelin antagonist, and a sex hormone, that is effective to ameliorate symptoms associated with a vascular condition. As used herein, the term “combination” of at least two agents selected from an anti-pressor agent, an endothelin antagonist, and a sex hormone means that at least two agents can be delivered in a simultaneous manner, in combination therapy wherein for example, an anti-pressor agent is administered first, followed by an endothelin antagonist, as well as wherein an endothelin antagonist is delivered first, followed by an anti-pressor agent. The desired result can be either a subjective relief of a symptom(s) or an objectively identifiable improvement in the recipient of the dosage.

The term “inhibitor of phosphodiesterases” (PDE) is an agent that can either activate or suppress PDEs via allosteric interaction with the enzymes or binding to the active site of the enzymes. The PDE family includes at least 19 different genes and at least 11 PDE isozyme families, with over 50 isozymes having been identified thus far. The PDEs are distinguished by (a) substrate specificity, i.e., cGMP-specific, cAMP-specific or nonspecific PDEs, (b) tissue, cellular or even sub-cellular distribution, and (c) regulation by distinct allosteric activators or inhibitors. PDE inhibitors include both nonspecific PDE inhibitors and specific PDE inhibitors (those that inhibit a single type of phosphodiesterase with little, if any, effect on any other type of phosphodiesterase). Still other useful PDE inhibitors are the dual selective PDE inhibitors (e.g., PDE III/V inhibitors or PDE II/IV inhibitors).

In one embodiment, the PDE inhibitor is a PDE V inhibitor. Useful phosphodiesterase type V inhibitors include, e.g., vardenafil, tadalafil, 1,3-dimethyl-6-(2-propoxy-5-methanesulfonylamidophenyl)pyrazolo[3, 4d]-pyrimidin-4-(5H)-one (DMPPO), cialis, vardenafil, tanadafil, zaprinast, MBCQ, MY-5445, dipyridamole, furoyl and benzofuroyl pyrroloquinolones, 2-(2-Methylpyridin-4-yl)methyl-4-(3,4,5-trimethoxyphenyl)-8-(pyrimidin-2-yl)methoxy-1,2-dihydro-1-oxo-2,7-naphthyridine-3-carboxylic acid methyl ester hydrochloride (T-0156), T-1032 (methyl 2-(4-aminophenyl)-1,2-dihydro-1-oxo-7-(2-pyridylmethoxy)-4-(3,4,5-trimethoxy-phenyl)-3-isoquinoline carboxylate sulfate), and sildenafil. Cyclic GMP specific inhibitors include but not limited to A02131-1 [3-(5′-hydroxymethyl-2′-furyl)-1-benzyl thieno (3,2-c)pyrazole] for example. In another embodiment, the composition contains a phosphodiesterase type II (PDE II) inhibitor such as, e.g., EHNA. In yet another embodiment, the composition contains a phosphodiesterase type IV (PDE IV) inhibitor. Suitable phosphodiesterase type IV inhibitors include, but are not limited to, roflumilast, ariflo (SB207499), RP73401, CDP840, rolipram, mesopram, denbufylline, EMD 95832/3, cilomilast, RO-20-1724, and LAS31025. In still another embodiment, the phosphodiesterase inhibitor is a dual selective phosphodiesterase inhibitor such as, e.g., a PDE III/IV inhibitor (e.g., zardaverine) or phosphodiesterase inhibitors which can increase both cAMP and cGMP levels such as Satigrel (E5510, 4-cyano-5,5-bis(4-methoxyphenyl)-4-pentenoic acid).

In another embodiment, the PDE inhibitor is an inhibitor of the PDE III isozyme, for example, Olprinone.

In another embodiment, the PDE inhibitor is an inhibitor of the PDE IV isozyme family, or cAMP-specific and rolipram sensitive PDEs, which preferentially hydrolyze cAMP.

In yet another embodiment, the composition contains an agent that is a nonspecific phosphodiesterase inhibitor. Suitable nonspecific phosphodiesterase inhibitors include, but are not limited to, theobromine, dyphylline, IBMX, theophylline, aminophylline, pentoxifylline, papaverine, caffeine and other methylxanthine derivatives.

The term “anti-pressor agent” as used herein denotes a therapeutic agent which acts either directly or indirectly to lower blood pressure. The term anti-pressor agent is chosen, rather than the more specific term “antihypertensive” agent, because the invention contemplates the use of agents which are effective to increase vascular flow in both hypertensive and normotensive patients. Anti-pressor agents contemplated for use in the method of the present invention include agents that act to bring about a lowering of blood pressure by any of a number of different physiological mechanisms. Anti-pressor agents include compounds belonging to a number of therapeutic classes based upon their mechanism of action, even though the therapeutic outcome is the same. Anti-pressor agents suitable for the method of this invention include compounds which are direct-acting vasodilators, such as hydralazine. Other suitable anti-pressor agents are compounds which act to inhibit the enzyme which converts the less potent decapeptide vasoconstrictor, angiotensin-I, to the more potent octapeptide vasoconstrictor, angiotensin II (so-called angiotensin-II converting enzyme inhibitors or “ACE inhibitors”), as well as agents which block the binding of angiotensin-II to the AT₁ receptor (“angiotensin-II receptor antagonists”). Anti-pressor agents useful in the method of the present invention also include vasodilating agents which act at α₁-adrenergic receptors or β-adrenergic receptors in the smooth muscle of vascular walls (“α₁-adrenergic receptor antagonists” and “β-adrenergic receptor antagonists”), as well as agents which act to decrease intracellular calcium ion concentration in arterial smooth muscle (“calcium channel blockers”). Suitable anti-pressor agents for use in the present invention also include activators of the enzymes guanylyl cyclase and adenyl cyclase, such as YC-1 and forskolin, respectively. PGE₁ (prostaglandin-E₁), which acts both as an anti-pressor agent and as a sexual response initiator, is also suitable for use in the invention. Also contemplated as falling within the scope of the invention for use as anti-pressor agents are phosphodiesterase inhibiting agents, particularly type-3 and type-5 phosphodiesterase inhibitors. Antagonists of PDE-5 (phosphodiesterase type 5), the enzyme primarily responsible for the degradation of cyclic guanosine monophosphate (cGMP), produce an increase in levels of cGMP, which, by way of “cross-talk,” also decreases the activity of PDE-3, the enzyme primarily responsible for the degradation of cyclic adenosine monophosphate (cAMP). Thus, increasing levels of cGMP acts to inhibit the PDE-3 enzyme, thereby blocking the degradation of cAMP and causing an increase in cAMP levels. Thus, inhibition of either PDE-5 or PDE-3 results in an overall increase in concentrations of cAMP and cGMP.

ACE inhibitors include benzazapine compounds such as benazepril and libenzapril; 6H-pyridazino[1,2a]diazepine derivatives such as cilazapril; 2,3-dihydro-1-indene compounds such as delapril; L-proline derivatives such as alacepril, captopril, ceronapril, enalapril, fosinopril, lisinopril, moveltipril, and spirapril; oxoimidazoline derivatives such as imidapril; 1,4-dihydropyridine compounds such as lacidipine; iso-quinoline carboxylic acid derivatives such as moexipril and quinapril; 1H-indole carboxylic acid derivatives such as pentopril and perindopril; hexahydroindole carboxylic acid derivatives such as trandolapril; cyclopenta[b]pyrrole carboxylic acid derivatives such as ramipril; and 1,4-thiazepine compounds such as temocapril.

Angiotensin-II receptor antagonists useful as anti-pressor agents in the method of this invention include eprosartan, irbesartan, losartan, and valsartan.

α₁-adrenergic receptor antagonists include substituted phenyl derivatives such as midrodrine, phenoxybenzamine, and tamsulosin; substituted naphthyl derivatives such as naphazoline; aminoquinazoline derivatives such as alfuzosin, bunazosin, doxazosin, prazosin, terazosin, and trimazosin; benzamide compounds such as labetolol; carbazole derivatives such as carvedilol; dimethyluracil derivatives such as urapidil; imidazolidinyl derivatives such as apraclonidine and clonidine; dihydroimidazole derivatives such as phentolamine; indole derivatives such as indoramin; and 1,2,4-triazolo[4,3-a]pyridine compounds such as dapiprazole.

Calcium channel blockers include benzothiazepine compounds such as diltiazem; dihydropyridine compounds such as nicardipine, nifedipine, and nimopidine; phenylalkylamine compounds such as verapamil; diarylaminopropylamine ether compounds such as bepridil; and benimidazole-substituted tetralin compounds such as mibrefadil.

Phosphodiesterase inhibitors include bipyridone compounds such as amrinone; and dihydropyrazolopyrimidine compounds such as sildenafil. Sildenafil functions as a selective type-5 (i.e. cGMP specific) phosphodiesterase inhibitor, and acts to decrease the metabolism of cGMP, the second messenger in nitric oxide mediated erectile response. An oral formulation of this medication has proven to be safe and effective in improving erectile duration and rigidity. In females, nitric oxide/NOS exists in human vaginal and clitoral tissue. Sildenafil, alone or in combination with other vasoactive agents, is effective for the long term management of sexual dysfunction for the treatment of vasculogenic male or female sexual dysfunction. Preferred PDE inhibitors include vardenafil, tadalafil, and 1,3-dimethyl-6-(2-propoxy-5-methanesulfonylamidophenyl)pyrazolo[3, 4d]-pyrimidin-4-(5H)-one (DMPPO).

The term “endothelin antagonist” as used herein denotes a therapeutic agent which acts either directly or indirectly to lower blood pressure. Endothelin antagonists contemplated for use in the method of the present invention include agents which act to bring about a lowering of blood pressure by any of a number of different physiological mechanisms. Endothelin antagonists include, but are not limited to, compounds belonging to a number of therapeutic classes based upon their mechanism of action, even though the therapeutic outcome is the same. Endothelin antagonists suitable for the method of this invention include peptide antagonists such as PD145065 (Parke Davis); non-peptide antagonists such as bosentan (Hoffman-LaRoche), astrasentan (Abbott), BQ-123, BQ-485, enrasentan, sitasentan, S-1255, TAK-044, tezosentan, ABT-546; inhibitors of endothelin converting enzyme (which block the production of endothelin), such as phosphoramidon; and antisense oligonucleotides which specifically block the translation of the endothelin protein. Those of skill in the art will know of other endothelin antagonists suitable for use in the present invention.

The term “sex hormone” refers to a human sex hormone, or salt, ester, derivative, agonist, antagonist, metabolite, mimetic, synthetic analog, or combination thereof, including, but not limited to, androgens, estrogens, and progestins. The sex hormone can also be a testosterone-like compound, estrogen-like compound, or progestin-like compound. Testosterone-like compounds include, but are not limited to, testosterone, testosterone propionate, testosterone enanthate, testosterone cypionate, testosterone undecenoate, dihydrotestosterone, danazol, fluoxymesterone, methyltestosterone, oxandrolone, DHEA, tibolone, and the pharmaceutically acceptable salts, esters derivatives, metabolites, mimetics, or synthetic analogs and mixtures thereof. Estrogen-like compounds include, but are not limited to, 17-β-estradiol, estrone, mestranol, estradiol valerate, estradiol cypionate, ethynyl estradiol, quinestrol, estrone sulfate, equilin, raloxifene, phytoestrogens including, but not limited to, flavones, isoflavones (e.g., genistein), resveratrol, coumestan derivatives, and the pharmaceutically acceptable salts, esters, derivatives, metabolites, mimetics, or synthetic analogs and mixtures thereof. Estrogen-like compounds include those compounds that bind to the estrogen receptor and act as agonists thereof. Progestin-like compounds include, but are not limited to, progesterone, hydroxyprogesterone caproate, medroxyprogesterone acetate, 19-nortestosterone, 19-norprogesterone, 17-OH-progesterone, norethynodrel, norgestrel, desogestrel, norgestimate, norethindrone (norlutin), norethindrone acetate (norlutate, aygestin), levonorgestrel, etonogestrel, gestodene, dienogest, drospirenone, trimegestone, nomegestrol acetate, and the pharmaceutically acceptable salts, esters, derivatives, metabolites, mimetics, or synthetic analogs and mixtures thereof.

### Pharmaceutical Compositions

Pharmaceutical compositions which are useful in the method of the present invention comprise one or more compounds defined above formulated together with one or more non-toxic pharmaceutically acceptable carriers. The pharmaceutical compositions may be specially formulated for oral administration in solid or liquid form, for parenteral injection, or for vaginal or rectal administration. The formulations may, for example, contain a single therapeutic agent selected from ACE inhibitors, angiotensin-1 (AT₁) receptor antagonists, α₁-adrenoreceptor antagonists, β-adrenergic receptor antagonists, direct-acting vasodilators, calcium channel blockers, phosphodiesterase inhibitors, or a combination of two or more agents selected from the same or different therapeutic categories. Moreover, a combination of one or more therapeutic agents from the groups listed above may be combined with a diuretic agent of the class well known in the art. The formulations may also contain, for example, a single endothelin antagonist such as a peptide antagonist, a non-peptide antagonist, an inhibitor of endothelin converting enzyme, or a combination of two or more antagonists selected from the same or different category. Further, the formulations may contain, for example, a single sex hormone such as an androgen, an estrogen, a progestin, or a combination of two or more hormones selected from the same or different category.

To enhance delivery to genital vasculature, combined systemic delivery with topical administration of an erectogenic initiator is also contemplated within the scope of this invention. In this manner the anti-pressor drug is delivered to target regions at a markedly enhanced rate. Since prostaglandin-E₁ acts both as an anti-pressor and as a direct sexual response initiator, one or more therapeutic agents from the groups listed above can be administered in combination therapy with prostaglandin PGE₁. The co-administered PGE₁ may be administered by any of the routes discussed below, with topical application being a preferred route.

The pharmaceutical compositions of this invention can be administered either systemically or locally to humans and other animals. Systemic routes include oral, parenteral, intracistemal, intraperitoneal, trans-cutaneous (by injection or patch), buccal, sub-lingual administration, or by means of an oral or nasal spray. Other methods include intracavemosal, implant, depot injection, topical transdermal and trans-mucosal. The term “parenteral” administration as used herein refers to modes of administration which include intravenous, intramuscular, intraperitoneal, intrastemal, subcutaneous and intraarterial injection, and infusion. Local administration routes include vaginal, rectal, intraurethral, trans-urethral, by intra-cavemosal injection, or topical administration.

Pharmaceutical compositions of this invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous careers, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

In some cases, in order to prolong the effect of the drug, it is desirable to slow the release or absorption of the drug following subcutaneous or intramuscular injection. This may be accomplished by the use of a liquid suspension of crystalline or amorphous material with low water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(othoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and 1) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, ground nut corn, germ olive, castor, and sesame oils), glycerol, tetrahydrofurfuryl alcohol, polyethylene glycols, fatty acid esters of sorbitan, and mixtures thereof. Besides inert diluents, the oral compositions can also include adjuvants such as wetting agents, emulsifying and suspending agents, sweetening, flavoring, and perfuming agents.

Suspensions, in addition to the active compound(s), may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Compositions for rectal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods for the formation of liposomes are known in the art. See, for example, Prescott, Ed., Methods in Cell Biology, Volume XIV, Academic Press, New York, N.Y. (1976), p. 33 et seq.

Actual dosage levels of active ingredients in the pharmaceutical compositions of this invention may be varied so as to obtain an amount of the active compound(s) that is effective to achieve the desired therapeutic response for a particular patient, composition, and mode of administration. The selected dosage level will depend upon the activity of the particular compound, the route of administration, the severity of the condition being treated, and the condition and prior medical history of the patient being treated. However, it is well known within the medical art to determine the proper dose for a particular patient by the “dose titration” method. In this method, the patient is started with a dose of the drug compound at a level lower than that required to achieve the desired therapeutic effect. The dose is then gradually increased until the desired effect is achieved. Starting dosage levels for an already commercially available therapeutic agent of the classes discussed above can be derived from the information already available on the dosages employed for the use of the compound as an antihypertensive agent.

In a repetitive dosing regimen to treat vascular conditions, a combination of at least two agents selected from an anti-pressor agent, an endothelial antagonist, and a sex hormone are co-administered for at least two treatment cycles separated by a drug-free period. In one embodiment, the co-administration of at least two agents occurs for at least two treatment cycles of at least 7 days, with each of the treatment cycles being separated by a drug-free period of at least 7 days. In another embodiment, the co-administration of at least two agents occurs for at least two treatment cycles of about 14 days, with each of the treatment cycles being separated by a drug-free period of about 14 days. Further, the duration of a combination treatment and a drug-free period can vary for each cycle. The treatment cycles can be from at least 7 days to about 21 days in duration. The drug-free periods can be from at least 7 days to about 21 days in duration.

In a preferred aspect, the present invention provides a method for the elimination or reduction of tolerance to an anti-pressor agent via the inclusion of an endothelin antagonist in a combination therapy approach to remodel vascular structure and treat vascular conditions associated with a male or female sexual dysfunction, atherosclerosis, renal failure, hypertension, congestive heart failure, diabetic nephropathy, and diabetic neuropathy, thereby enhancing the efficacy of treatment with an anti-pressor agent. Further, the co-administration of an anti-pressor agent and an endothelin antagonist enables a patient in need of such treatment for a vascular condition to receive higher doses or longer duration of an anti-pressor agent without the concomitant side-effects of decreased efficacy due to anti-pressor tolerance upon repeated administrations, thereby allowing for an increase in the number of therapeutically effective anti-pressor administrations. Thus, a combined therapy approach as such using an anti-pressor agent with an endothelin antagonist provides an efficacious therapeutic strategy for the long term treatment of vascular-conditions that is lacking in the art.

For the preferred therapeutic anti-pressor agents in the method of the present invention, namely ACE inhibitors and angiotensin-II receptor antagonists, generally dosage levels of about 0.1 mg to about 300 mg, more preferably of about 0.5 mg to about 150 mg of active compound per kilogram of body weight per day are administered orally to a patient, with the dose levels appropriately adjusted if the route of administration is other than oral. If desired, the effective daily dose may be divided into multiple doses for purposes of administration, e.g. two to four separate doses per day.

### Biological Data

A. Demonstration that the Sex Organs are Not Protected from Pathological Vascular Degradative Modeling

1. Methodology

Male spontaneously hypertensive (SH) rats weighing between 246-313 g and normotensive Sprague-Dawley (SD) rats weighing between 246-440 g were obtained from Charles River Laboratories (Montreal, Quebec, Canada). The animals were maintained in individual cages with a 12 hour light/12 hour dark cycle, and a room temperature of 22-24° C. They were provided with standard rodent chow and tap water ad libitum and were acclimated to the room for at least two days before the experiments. All procedures were carried out in accordance with the guidelines set out by the Canadian Council on Animal Care.

2. Penile Vascular Resistance Properties

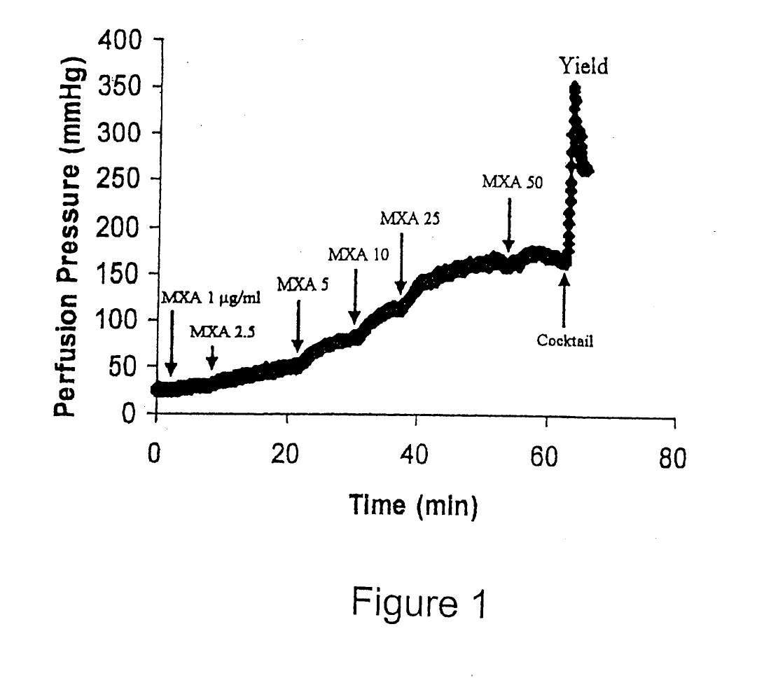

Penile perfusion preparations were made using the procedure established by Banting, J. D., et al., “Isolation and Perfusion of the Pudendal Vasculature in Male Rats. J Urol., 2: 587-590 (1995). A heated chamber served to maintain the ambient temperature and the entire preparation at 37-38° C. The perfusate was held in a reservoir and passed through a heating and a bubble trapping/mixing chamber connected to a single peristaltic pump (Minipuls 2 Pump, Gilson, Inc., 3000 W. Beltline Highway, Middleton, Wis. USA 53562). An injection port was located distal to the pump for the introduction of pharmacological agents to minimize dead volume. Drugs were administered via a Harvard Apparatus, Inc. infusion pump (Harvard Apparatus, Inc., 84 October Hill Road, Holliston, Mass. 01746). The perfusate was a Tyrode-dextran solution consisting of a mixture of 20 mg of KCl, 32.3 mg of CaCl 2.H₂O, 5.1 mg of MgCl₂.6H₂O, 6.2 mg of NaH2PO₄.2H₂O, 155 mg of NaHCO₃, 100 mg of glucose, and 800 mg of NaCl in each 100 mL of fluid. The solution was maintained at pH 7.4, and a temperature of 37-39° C., and oxygenated with 95% O₂ and 5% CO₂. The rats were anaesthetized (sodium pentobarbital 60 mg/kg body weight i.p.) and heparinized (1000 IU/kg, i.v.). The bilateral isolation of penile vasculature was achieved by ligating all of the branching arteries except for the pudendal; then the abdominal aorta was cannulated proximal to the iliac bifurcation with a single lumen catheter. The catheter was connected to the perfusion apparatus via a pressure transducer for arterial pressure recording. After sectioning the vena cava and spinal cord to remove venous resistance and to eliminate neural influences, a flow of perfusate (1 mL/min per kg body weight) through the abdominal cannula was initiated. The perfusion pressure was continuously recorded on a data acquisition system (MacLab, AD Instruments, Houston, Tex.). The perfusate was infused for twenty minutes to flush the penile vasculature of blood and obtain a stable pressure before the beginning of any experiment. Following this, sodium nitroprusside (20 g/mL) was infused to induce maximum vasodilation. The flow rate-perfusion/pressure relationship was determined by measuring the pressure at minimum vascular resistance at flow rates of 0.5, 1.0, 2.0, 4.0 mL/min per kg of body weight. A cumulative α₁-adrenoreceptor concentration-response curve to methoxamine (2.5, 5, 10, 25, 50 g/mL was then generated. Each concentration of methoxamine was infused for a duration of 10 minutes, at which time a plateau was reached. Subsequently, a continuous injection of a cocktail containing a supramaximal concentration of vasoconstrictors (vasopressin, 20.5 g/mL, angiotensin-II, 200 ng/mL; methoxamine, 64 g/mL; Sigma, St. Louis, Mo., 63178) was given to ensure that maximum constrictor response that was not dependent upon the activation of a single receptor type was achieved. A second injection of the constrictor cocktail was administered to ensure the tissue “yield” was maximum constriction. This “yield” induced by the multi-vasoconstrictor cocktail has been demonstrated to correlate directly with the bulk of medial vascular smooth muscle cells in the resistance vasculature. A typical perfusion pressure tracing from this protocol can be seen in FIG. 1. At the end of the concentration-response relationship, the aorta was cut distal to the catheter, and a baseline flow-pressure curve was recorded again. This was done to ensure that pressure fell to zero and to account for any false pressure readings that may have resulted due to movement of the catheter during the experiment.

3. Hindlimb Vascular Resistance Properties

The hindlimb perfusion preparation was adopted from a technique originally designed by Folkow et al., Acta Physiol Scand., 80: 93-106 (1973), as modified by Adams et al., Hypertension 14: 191-202 (1989). The perfusion experiment was performed as described above. Drugs were administered into the mixing chamber via a Harvard Apparatus infusion pump. The rats were anaesthetized (Inactin, 100 mg/kg of body weight, i.p.) and heparinized (1000 IU/kg of body weight, i.v.). Following a midline abdominal incision, the abdominal aorta was cannulated proximal to the iliac bifurcation with a double lumen catheter (Storz, St Louis, Mo., USA), and the catheter was extended down the right common iliac artery. One lumen of the catheter was connected to the perfusion apparatus, while the other was connected to a pressure transducer for arterial pressure recording. The rat was perfused at a constant flow rate (2 mL/min per 100 g of body weight) and the experiments were carried out as described above. The flow rate/perfusion pressure relationship was recorded at flow rates of 0.5, 1.0, 2.0, 4.0 mL/min per 100 g of body weight. A cumulative α₁-adrenoreceptor concentration-response curve to methoxamine (0.5, 1, 2, 4, 8, 16, 32, 64 g/mL) was then generated. Each concentration of methoxamine was infused for a duration of 5 minutes, at which time a plateau was reached. Subsequently, a bolus injection of a cocktail containing a supramaximal concentration of vasoconstrictors was given as above. At the end of the concentration-response relationship, the iliac artery was cut distal to the catheter, and the flow pressure curve was monitored again.

Flow rates for the hindlimb perfusion experiments were determined based on expected flow rates of exercising skeletal muscle at maximum dilation. The flow rate used resulted in a perfusion pressure at maximum dilation between 20-25 mm Hg which is well within the expected range. After checking several flow rates in the penile perfusion, a rate was obtained that resulted in a similar perfusion pressure at maximum dilation. The flow rates chosen also allow for the assessment at maximum constriction. This allowed for comparison between strains.

4. Analysis of Data

All values in the figures and tables were expressed as mean.+−.standard deviation. Results comparing penile and hindlimb vasculature were analyzed using the Student's t-test. Differences were considered as significant at p<0.05.

5. Results

There was no significant difference in the body weight of the spontaneously hypertensive rats in the penile assessment group (267.+−.29 g, n=5) and in the hindlimb assessment group (270.+−.5.7 g, n=3). The average body weight of the normotensive Sprague-Dawley rats in the hindlimb assessment group (375.+−.41 g, n=8) was significantly higher than that of the of the normotensive Sprague-Dawley rats in the penile assessment group (284.+−.32 g, n=5). However, this was not considered relevant because in normotensive adult rats there has been shown to be very little correlation between body weight and blood pressure (Adams, M. A., et al., Hypertension, 14:191-202 (1989).

This hemodynamic analysis had similar effects in most parameters between the penile and hindlimb vascular beds within each rat strain. The flow-pressure curve assessed at maximal dilation was similar in both the penile and the hindlimb vasculatures of spontaneously hypertensive and normotensive rats as shown in Table 1. These curves were monitored to ensure a linear increase in perfusion pressure with an increase in flow rate. The increase in the flow rate exerted a radial pressure against the vessel wall and resulted in increased pressure. The spontaneously hypertensive rats trended towards a higher baseline pressure than the normotensive rats. This was observed in both penile and hindlimb vascular beds. These data suggest that spontaneously hypertensive rats may have a smaller lumen thus causing them to operate at a higher pressure than normotensive Sprague-Dawley rats even when there is no constrictor tone on the vessel.

TABLE 1

| | | Maximum | | |
|---|---|---|---|---|
| | | Constriction | | |
| | Slope | With | | |
| | Flow | Methoxamine | | Slope |
| Group | Pressure | (mm Hg) | Log EC₅₀ | Methoxamine |
| SHR, | 7.15 ± 2.0 | 172 ± 32 | 0.95 ± 0.19 | 1.64 ± 0.21 |
| penile | | | | |
| bed | | | | |
| SHR, | 6.68 ± 0.38 | 253 ± 25* | 0.79 ± 0.15 | 5.19 ± 3.0* |
| hindlimb | | | | |
| bed | | | | |
| SD, | 7.34 ± 2.3 | 171 ± 36 | 0.63 ± 0.24 | 2.03 ± 0.68 |
| penile | | | | |
| bed | | | | |
| SD, | 6.99 ± 3.4 | 191 ± 55 | 0.78 ± 0.12 | 3.0 ± 0.99 |
| hindlimb | | | | |
| bed | | | | |

*Statistically significant.

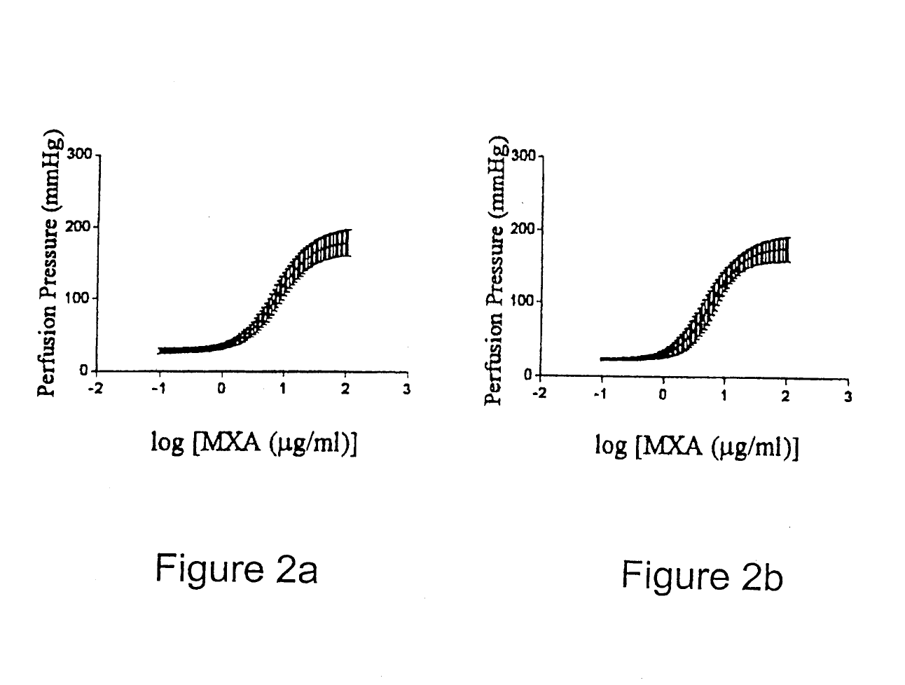

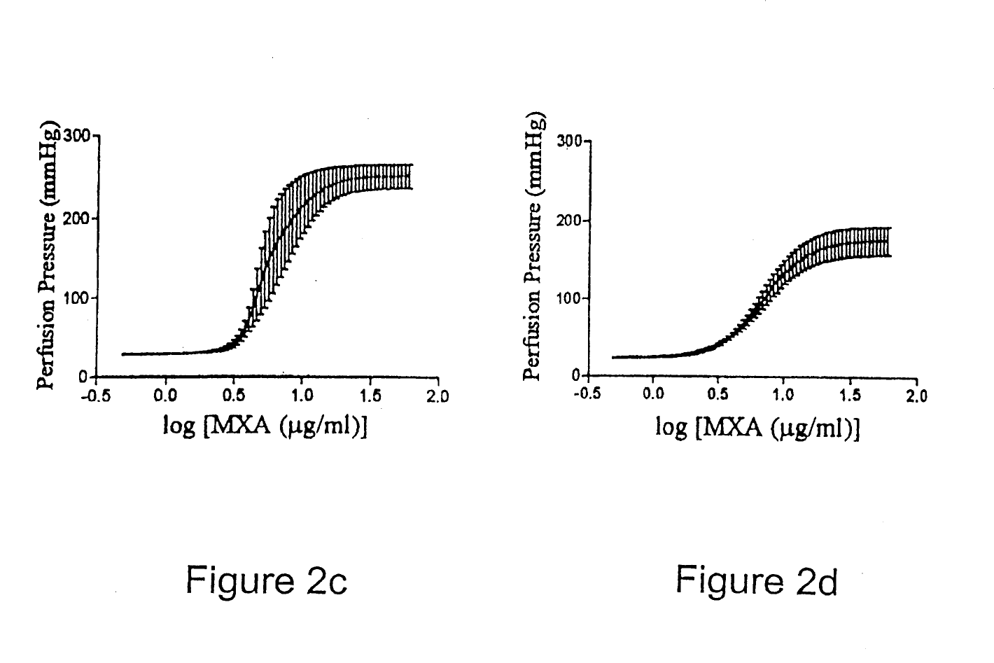

Table 1 shows there was a statistically significant difference in maximum constriction with a supramaximal dose of methoxamine (50 g/mL for penile and 64 g/mL for hindlimb vasculature) between spontaneously hypertensive rat hindlimb vasculature (253.+−.25 mmHg) and spontaneously hypertensive rat penile vasculature (172.+−.32 mmHg). This difference was not observed in normotensive Sprague-Dawley rats. The discrepancy is novel and requires further assessment. It is expected that higher responses would be seen in the spontaneously hypertensive rats in both arterial beds, however only the hindlimb vasculature showed a significant difference between spontaneously hypertensive and normotensive rats. Average concentration response curves for methoxamine of the two strains in both beds are shown in FIGS. 2a-2d.

The EC₅₀ of the concentration response curve shown in Table 1 gives the concentration of drug at which there is a 50% response to α₁ adrenoreceptor stimulation. This value would be an indication of the sensitivity of the tissue to this receptor activation. The logs EC₅₀ of the methoxamine concentration-response curves were not different for penile and hindlimb vasculature in both the spontaneously hypertensive and normotensive rats thus indicating similar sensitivity to this receptor stimulation.

The steepest slope of this curve is given in Table 1. In normotensive rats, there was no statistically significant difference in slope between penile vasculature (2.03.+−.0.68) and hindlimb vasculature (3.0.+−.0.99). The parameters showed a statistically significant difference between spontaneously hypertensive rat penile (1.64.+−.0.21) and spontaneously hypertensive rat hindlimb (5.19.+−.3.0). This was expected since the maximal constriction with methoxamine was lower in penile vasculature while the EC₅₀, remained the same.

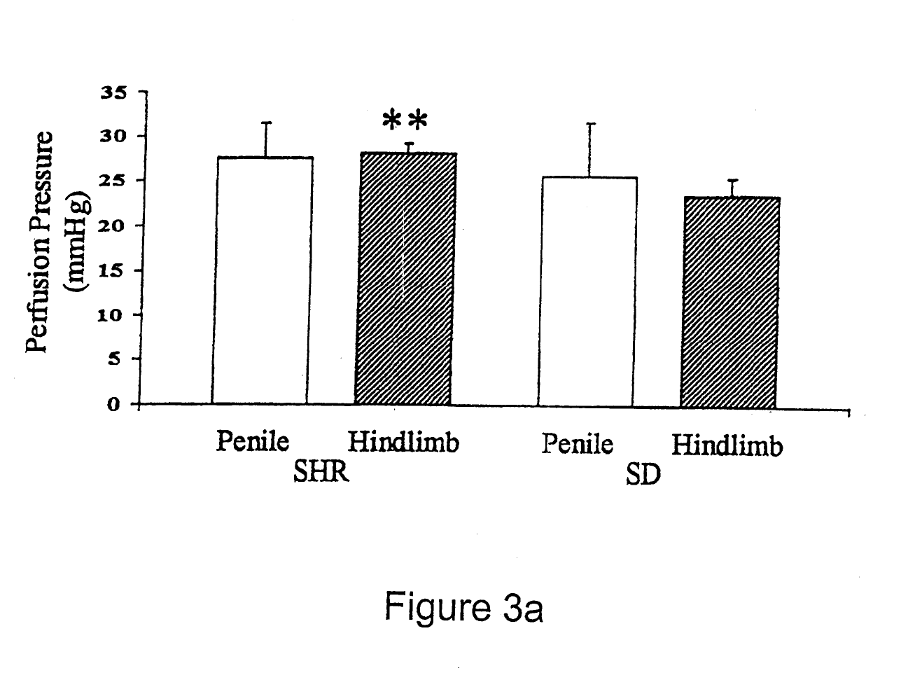

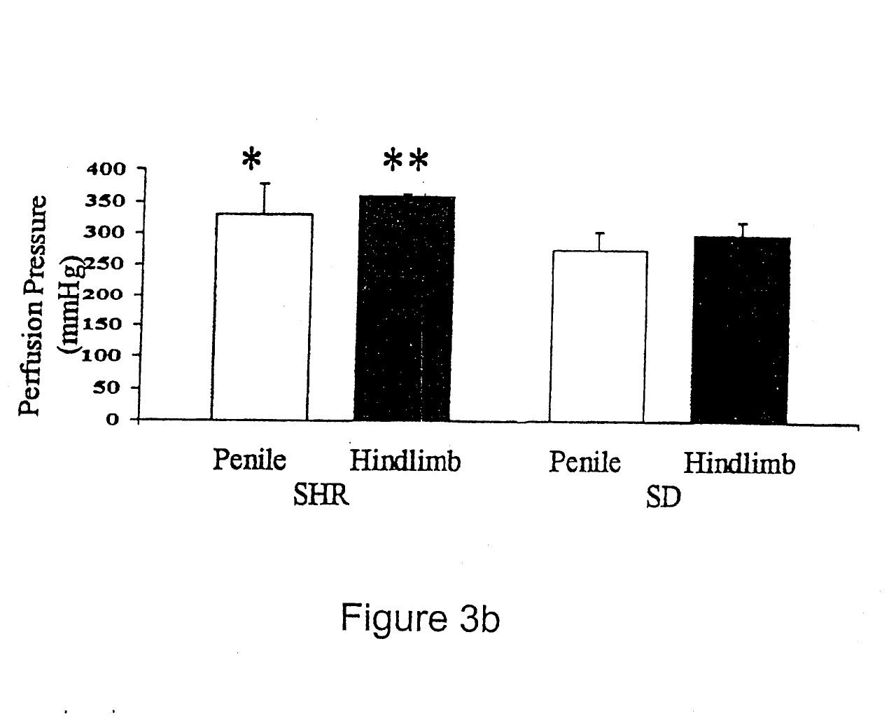

FIG. 3 depicts the structurally-based vascular resistance properties assessed at both maximum dilation and maximum constriction. There was no significant difference in perfusion pressures at maximum dilation within the rat strains. Between the two strains of rats, the penile vasculature trended towards higher pressures in the hypertensive rat as compared to the normotensive Sprague-Dawley rats, however it did not reach a level of significance as in the hindlimb. Spontaneously hypertensive rats reached a point of maximal constriction with a cocktail at a perfusion pressure that was 20% higher than normotensive rats in each vascular bed. There was no statistically significant difference between the two beds within strain, suggesting that the penile and hindlimb vasculature undergo similar structural changes in genetically hypertensive rats. This point demonstrates the increased medial thickening that occurs in the hypertensive rats that allows for the maintenance of higher arterial operating pressures.

6. Discussion

The major findings of the data presented above demonstrate that the penile vasculature is not protected from the structural changes that take place in the other vascular beds of spontaneously hypertensive rats relative to normotensive strains. Increased medial thickening and narrowing of the vascular lumen have been found in blood vessels of a wide range of vascular beds of spontaneously hypertensive rats. Therefore, the overall results of the present series of experiments have shown that the genetic disposition appears to dominate the structure regardless of the vascular bed.

In the present study a hemodynamic methodology was used to compare and contrast structurally-based vascular resistance in two vascular beds. The hindlimb bed was chosen for comparison since the vascular resistance properties are well established and anatomically the feeder vessels of the two beds are common.

These results demonstrate that the resistance properties at maximum dilation were similar in the two beds within strains. A general finding of studies comparing vascular resistance at minimum tone is that a higher perfusion pressure is normally obtained in SHR compared to normotensive rats. Thus, findings of elevated resistance properties at maximum dilation are consistent with there being an overall narrowing of the vascular lumen. Resistance properties were further assessed by determining the slope of the flow-pressure curve at maximum dilation. This relationship was used to determine whether there were any differences in the passive vascular wall elements such as the extracellular matrix components, i.e. if distensibility was altered there would be a differential effect on the flow-pressure curves. Further, a thicker medial wall could also result in a stiffer vessel which would exhibit less compliance with increasing flow. The lack of difference in all of these values suggests that there has been no differential change in the components of the vessel wall within the penile vasculature.

Assessment of the active components of the vessel walls was determined by inducing a state of maximal vasoconstrictor tone using a cocktail of vasoconstrictor agonists. The supramaximal, multiple agonist stimulus produces a maximum constrictor response which is independent of individual receptor population changes thereby reflecting only the overall contractile bulk of the medial smooth muscle cells.

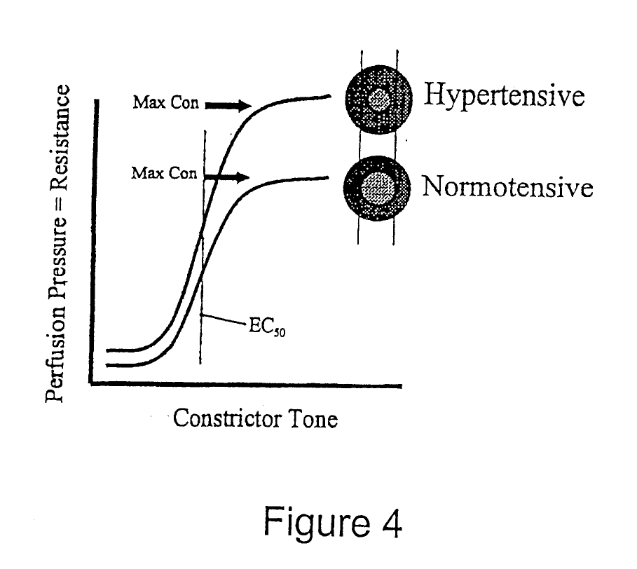

The findings that sensitivity (EC₅₀) and reactivity (slope) to α₁-adrenoreceptor stimulation were not different between vascular beds or strains likely indicates that there is a similar stimulus-response coupling of the noradrenergic innervation in all of these vessels; i.e. there is a consensus of normal vascular biology. In the schematic diagram of FIG. 4, the concept of structural changes dominating function is depicted. Thus, the curves show that, at any given level of vasoconstrictor tone, the hypertensive circulation will always have increased vascular resistance compared to normotensive circulation. Another way of looking at this concept would be that at the same level of constrictor tone, the normotensive circulation would be able to achieve the same inflow at a proportionately lower perfusion pressure than the hypertensive circulation.

Taken together, the findings indicate that the penile vasculature has an increased average medial mass coupled with decreased average lumen. The generation of an erection is based on the flow when vessels are in a dilated state. Although there was a significant difference in the perfusion pressure at maximum dilation in the hindlimb vasculature of spontaneously hypertensive rats (SHR) when compared to the normotensive Sprague-Dawley (SD) rats, this was not detected in the penile vascular bed. There was however a trend toward significance which may be seen in future studies when animals are used that are genetically closer to the spontaneously hypertensive rat, such as the Wistar-Kyoto rat (Taconic, 273 Hover Avenue, Germantown, N.Y. 12526) which is a more appropriate normotensive control. The penile vasculature is more complex than that of the hindlimb and therefore it may be that differences at maximum dilation are more difficult to detect. It is also unknown whether the size of the penis differs between the strains examined in this study. The length of the vessels changes baseline resistance more than the maximum constriction response because the maximum dilation is flow-dependent, as there is no constrictor tone on the vessel. In contrast, maximum constriction responses are based on the bulk muscular tissue. Therefore although the point of maximum dilation is expected to be higher in the penile vasculature of SHR as compared to a normotensive control it may not be detectable using the Sprague-Dawley rats as a comparison based on the suspected differences between the strains.

B. Therapeutically-Induced Vascular Remodeling in Penile Vasculature

1. Methodology

Adult spontaneously hypertensive rats (SHR) were treated for 1 or two weeks with either enalapril (30 mg/kg of body weight per day) or hydralazine (45 mg/kg of body weight per day). Following this treatment, structurally-based resistance to blood flow in the perfused penile vascular bed and hindlimb vascular bed models were measured using the methods detailed above. Cumulative α₁-adrenoreceptor concentration-response curves in response to methoxamine were measured as described above, and the “yield” point determined, following the achievement of maximal vasoconstriction using the vasopressin, angiotensin-II, methoxamine cocktail described above. The animal hearts were excised and weighed. The data are presented in Table 2 below.

2. Results

TABLE 2

| | | | | Left |
|---|---|---|---|---|
| | | Maximum | | Ventricle |
| | | Constriction | | Weight |
| | Slope | With Cocktail | | (g)/Body |
| | Flow | “Yield” (mm | | Weight (kg) |
| Group | Pressure | Hg) | Log EC₅₀ | Ratio |
| SHR-E₂¹ | 6.45 ± 1.31 | 335 ± 23 | 8.73 ± 0.26 | 2.13 ± 0.08 |
| (n = 7) | | | | |
| SHR-E₁¹ | 6.10 ± 1.5 | 358 ± 20 | 7.33 ± 1.39 | 2.17 ± 0.05 |
| (n = 5) | | | | |
| SHR-H² | 6.6 ± 0.86 | 353 ± 11 | 13.56 ± 5 | 2.37 ± 0.12 |
| (n = 7) | | | | |
| Control | 7.13 ± 0.63 | 381 ± 21 | 11.95 ± 5.51 | 2.46 ± 0.08 |
| (n = 9) | | | | |

¹Enalapril-treated animals.
²Hydralazine-treated animals

The data appearing in Table 2 show that enalapril treatment progressively regressed (remodeled) cardiac and pudendal vascular structure over the 2-week period of treatment. The “yield” value decreased on average by 12.1%.+−.6.0%, while left ventricular mass decreased by 13.6%.+−.3.2%. Hydralazine treatment was somewhat less effective, decreasing the “yield” point by 7.3%.+−.2.9%, and had no significant effect on left ventricular weight (decreased of 3.7%.+−.5.0%).

C. Evidence for an Inhibitory Role for Endothelin in the Persistent Response to Antihypertensive Therapy.

1. Methodology

Ten week old male spontaneously hypertensive rats (SHR, n=29) were obtained from Charles River Laboratories (Montreal, Canada) and housed individually in a temperature controlled (21±1° C.) room with a 12 hour light/dark cycle. Access to food (Purina rodent chow, 0.4% Na⁺) and water was ad libitum. All experimental procedures were approved by the Queen's University Animal Care Committee in accordance with guidelines established by the Canadian Council on Animal Care.

2. Drug Treatments

Animals were randomized to the following groups: enalapril (30 mg·kg⁻¹day⁻¹, n=5; Sigma Chemical Co., St. Louis, Mo., USA) and losartan (30 mg·kg⁻¹day⁻¹, n=5; Merck-Frosst Canada Inc., Point-Claire, Quebec), doses which have previously shown to produce equivalent depressor responses. Animals received the drugs in the drinking water (tap water). Drug concentrations in the drinking water were adjusted weekly to account for changes in animal weight or fluid intake. Treatment was initiated at 15 weeks of age, and drugs were administered for 3 treatment cycles of 2 weeks in duration. Treatment cycles were separated by 2-week drug-free periods. Throughout the study all diet regimens, access to food and fluid was ad libitum.

3. Mean Arterial Pressure Assessment

At 12 weeks of age, under isofluorane (dosed to effect, by inhalation, Janssen, North York, ON) anesthesia, radio-telemetric pressure transducers (model TA11PA-C40; Data Sciences Inc., St Paul, Minn., USA) were implanted into the abdominal aorta of 12 SHR using a previously described technique. Animals were appropriately recovered from surgery for 1 week before starting baseline data collection. Baseline data was collected for 1 week prior to initiation of drug treatments.

Throughout the entire study, mean arterial pressures (MAP) of each animal were collected continuously at 500 Hz for 30 seconds every 5 minutes, with each 30-second interval being stored as a single pooled value using the Dataquest software program (288 data points per day for each rat). The data from an age-matched group of control SHR (n=12) were used for comparison.

4. Assessment of the MAP-sodium Balance Relationship In Vivo

The MAP-sodium balance relationship was assessed in the off-treatment period (3-6 weeks off-treatment). Animals were administered 3 days of low salt diet (LS=Purina rodent chow, 0.04% Na⁺ and water), 5 days of a high salt diet (HS=Purina rodent chow 0.4% Na⁺ and water containing 1% NaCl), and 3 days of losartan treatment. This was done to compare the activity of the RAS between previous enalapril and losartan treatment and age-matched controls. Continuous MAP values were determined throughout these manipulations.

5. Hindlimb Vascular Resistance Properties

In a separate group of age-matched control (tap water, n=7) and treated (losartan 30 mg·kg⁻¹day⁻¹, n=10) SHR, structurally-based vascular resistance properties in the hindlimb were assessed at the end of the first 2-week treatment cycle (day 14). The hindlimb perfusion preparation was based on the technique originally designed by Folkow et al., Acta Physiol. Scand., 80:93-106 (1973), later modified by Adams et al., Hypertension 14:191-202 (1989). Briefly, in anesthetized (thiobutabarbital sodium 100 mg/kg BW i.p.) and heparinized (1000 IU/kg i.v.) rats, the right hindquarter was perfused with a double lumen catheter (Storz, St Louis, Mo., USA), which allowed for both perfusion and recording of perfusion pressure (MacLab, AD Instruments, Houston, Tex.). After sectioning the vena cava and spinal cord to remove venous resistance and to eliminate neural influences, the rat was perfused at a constant flow rate (2 mL/min per 100 g BW). The vasculature was perfused (20 minutes) to clear all blood and obtain a stable pressure prior to infusing sodium nitroprusside (SNP 20 μg/mL) to ensure that maximum vasodilatition had been established. The flow rate-perfusion pressure relationship was determined at flow rates between 0.5 and 4.0 mL/min per 100 g body weight (BW). A cumulative α₁-adrenoceptor concentration-response curve to methoxamine (MXA, 0.5-64 μg/mL) was then generated at 2.0 mL/min per 100 g BW. Subsequently, two sequential bolus injections of a cocktail containing a supramaximal concentration of vasoconstrictors (vasopression, 21 μg/mL; Ang II, 200 mg/mL; MXA, 64 μg/mL; Sigma) was given to produce a maximum response (i.e. “yield” response) that was not dependent upon the activation of a single receptor type. This “yield” response correlates with the bulk of medial vascular smooth muscle in the resistance vasculature.

6. Assessment of Left Ventricular Mass

Hearts were excised and blotted dry, right ventricle and left ventricle plus septum were then separated and weighed. Analysis of the left ventricle to body weight ratio was used as an index of change in cardiac structure.

7. Measurement of Endothelin Levels in the Kidney

Adult SHR were treated (enalapril 30 mg kg⁻¹ per day +0.04% Na⁺ diet, 2 wks) followed by a 2 wk drug free period. This on/off treatment pattern was repeated (total of 3 cycles). MAP was continuously monitored using radiotelemetry. Kidneys were excised prior to treatment, and on day 10 of the 1^{st} and 3^{rd} cycles of treatment. Renal cortex and medulla were separated and endothelin levels were measured on each section by radioimmunoassay.

8. Statistical Analysis

All values in the figures and tables are expressed as mean ± standard deviation. Data was analyzed for between group comparisons using a one-way analysis of variance with the Neuman Keuls post hoc test (Graph Pad Prism software). For within group analysis of MAP data were analyzed using the paired Student's T-Test. Hindlimb comparisons between losartan and control animals were analyzed using the unpaired Student's T-test. Differences were considered as significant at p<0.05.

9. Results

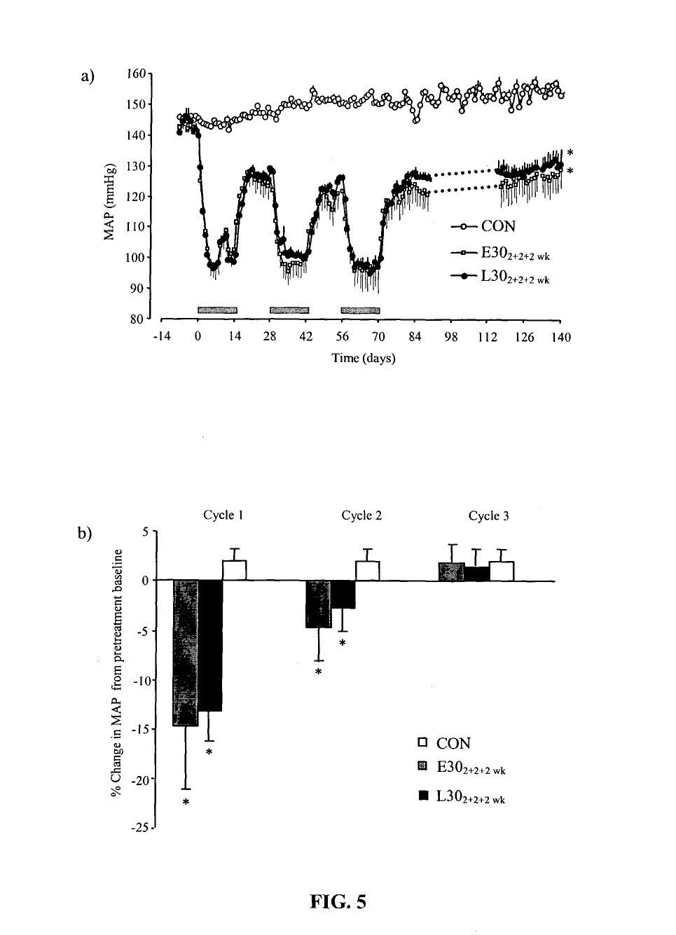

FIG. 5a illustrates the mean arterial pressure (MAP) profiles for animals treated with three cycles of either enalapril or losartan, and control animals. At the start of the study, the average MAP in enalapril (143±3.8 mmHg) and losartan (143±6.2 mmHg) treated animals were similar. There was a significant persistent effect of treatment on MAP over the last three weeks (days 120-140) of the study. FIG. 5b shows that during each cycle of treatment, MAP was similarly decreased with both enalapril (cycle 1: −28+3.8%, cycle 2: −21±3.4%, cycle 3: −20±5.8%) and losartan (cycle 1: −29±1.9%, cycle 2: −21±2.5%, cycle 3: −21±3.9%) treatments. The magnitude of persistent lowering of MAP was similarly diminished with each successive cycle for both enalapril (cycle 1: −15±6.4%, cycle 2: −5±4.3%, cycle 3: +1.5±1.49% vs. pretreatment) and losartan (cycle 1: −13±2.9%, cycle 2: −3+2.2%, cycle 3: +1.3±1.69% vs. pretreatment). The third cycle of treatment was not able to prevent the age related increase in MAP, as the change in blood pressure over that time frame was similar to that observed in control animals (+1.9±1.26%).

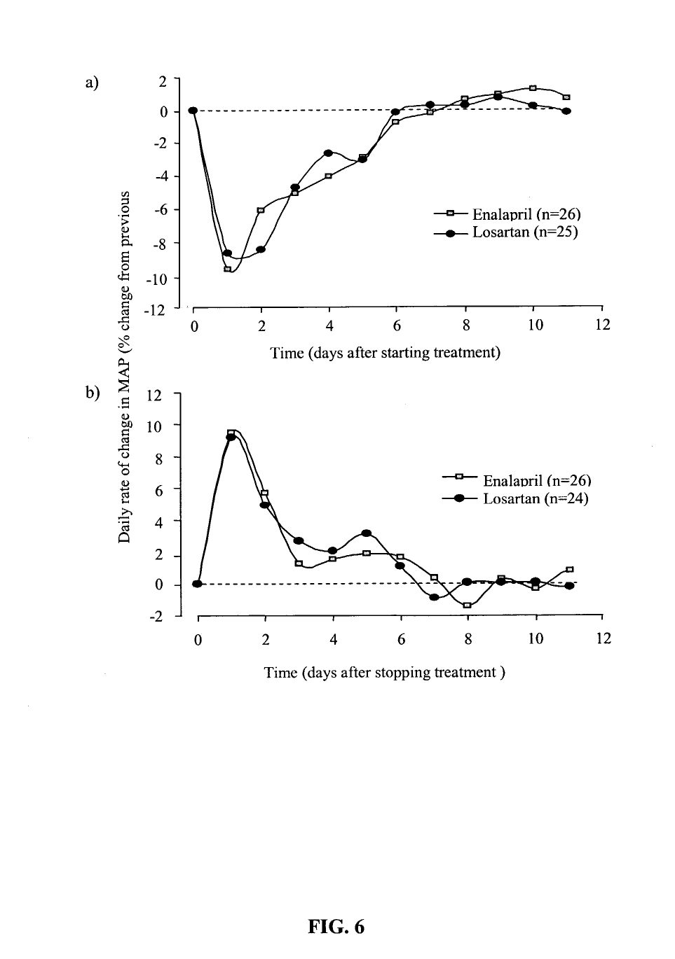

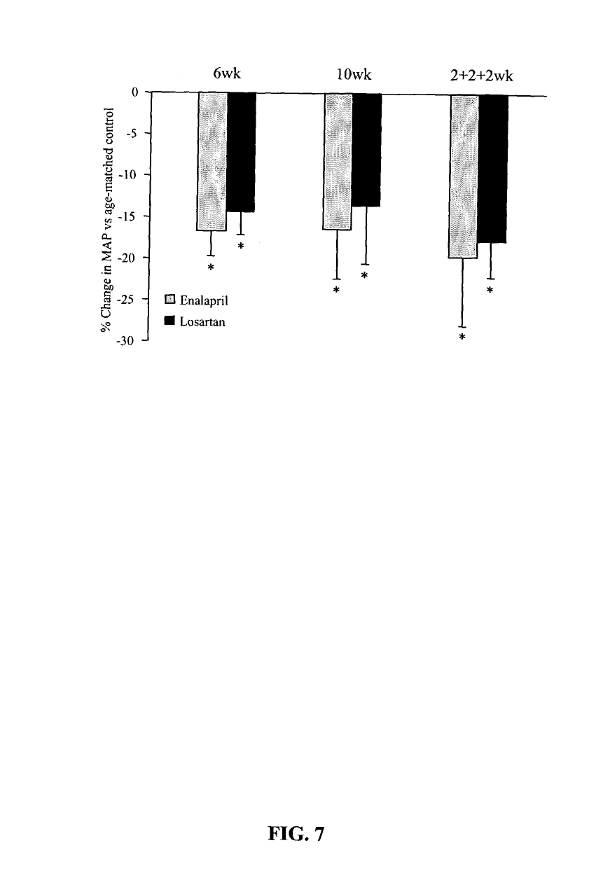

FIGS. 6a and 6b are graphs illustrating that additional analysis of the blood pressure profiles from animals treated with enalapril and losartan for up to 10 weeks revealed that the same level of pressure is achieved with these two agents throughout all treatment periods. The pattern of blood pressure change was also similar between antihypertensive agents, for example, in all cases steady state is achieved by the sixth day following the initiation of treatment. Similarly, when treatment has stopped, a new steady state is reached by the sixth day. Furthermore, FIG. 7 shows that analysis of the duration dependence of the persistent lowering response revealed that 6 weeks, 10 weeks, and the 2+2+2 week protocol all resulted in a similar degree of blood pressure lowering as compared to untreated SHR.

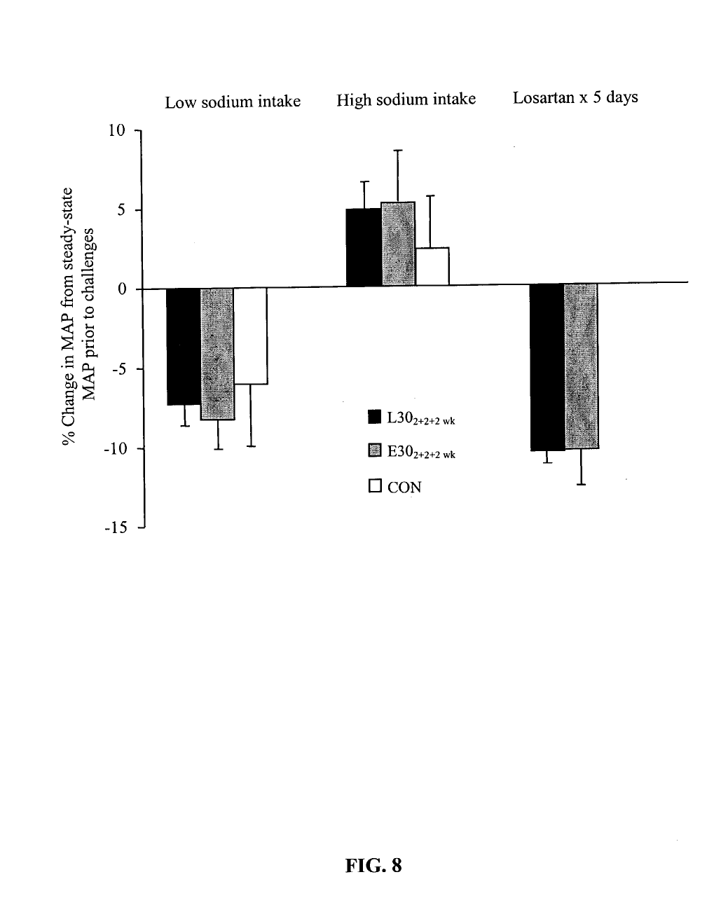

FIG. 8 illustrates that the sensitivity of the mean arterial pressure (MAP) to changes in dietary sodium intake was not significantly different between enalapril, losartan or control during LS diet (−7%) or HS diet (4%). Enalapril and losartan treated animals also had a similar effect when given a 5-day challenge to losartan (30 mg/kg per day).

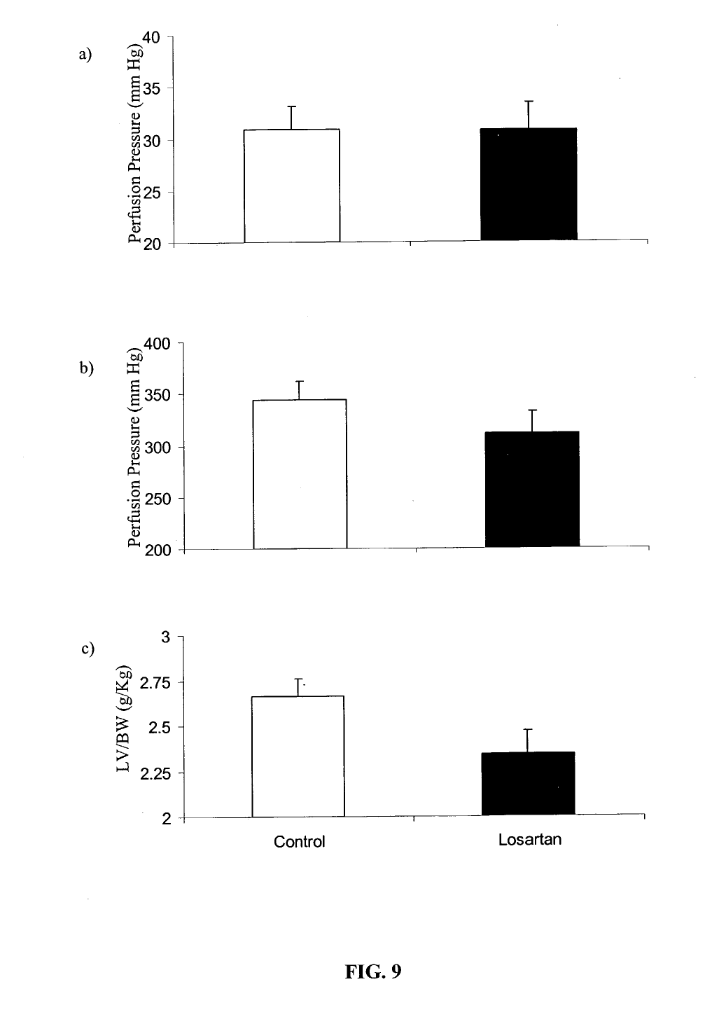

FIGS. 9a and 9b are graphs showing that two weeks of treatment with losartan induced a regression of structurally-based vascular resistance; however, this change was only evident at maximum constriction (Max Dil, ↓ 0.4±8.78%, p>0.05; Max Con, ↓ 9±6.31%, p<0.05) (Table 1). Treatment also induced a significant reduction in left ventricular mass (↓ 12±4.8%, p<0.05), as shown in FIG. 9c. However, there was no significant change in the maximum response to α₁-adrenoceptor stimulation, reactivity of this vasculature and there was no shift in the EC₅₀ of the α₁-adrenoceptor concentration-response curve.

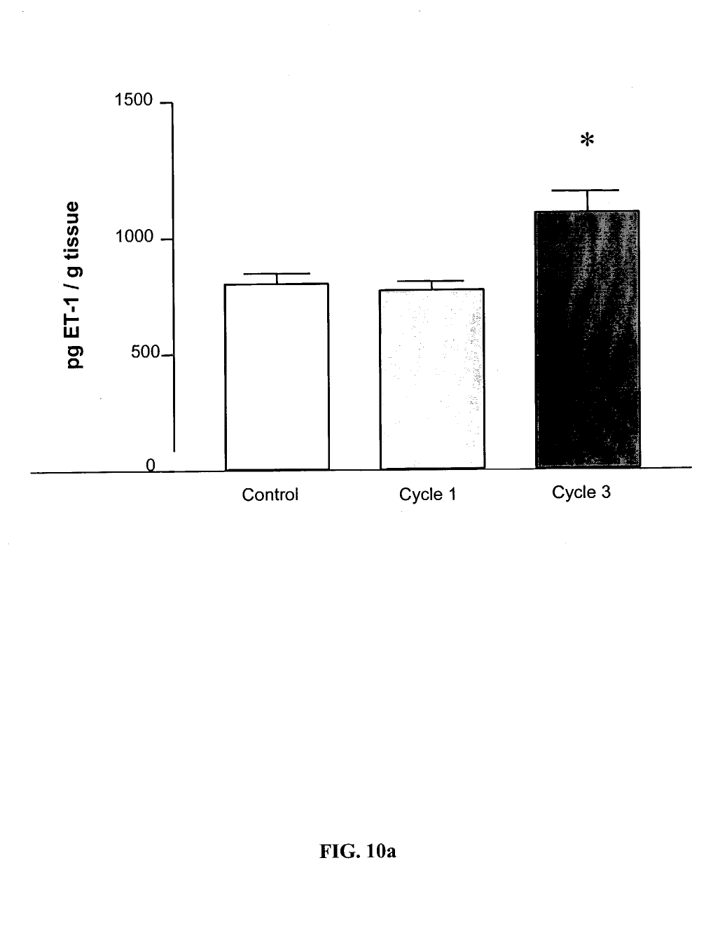

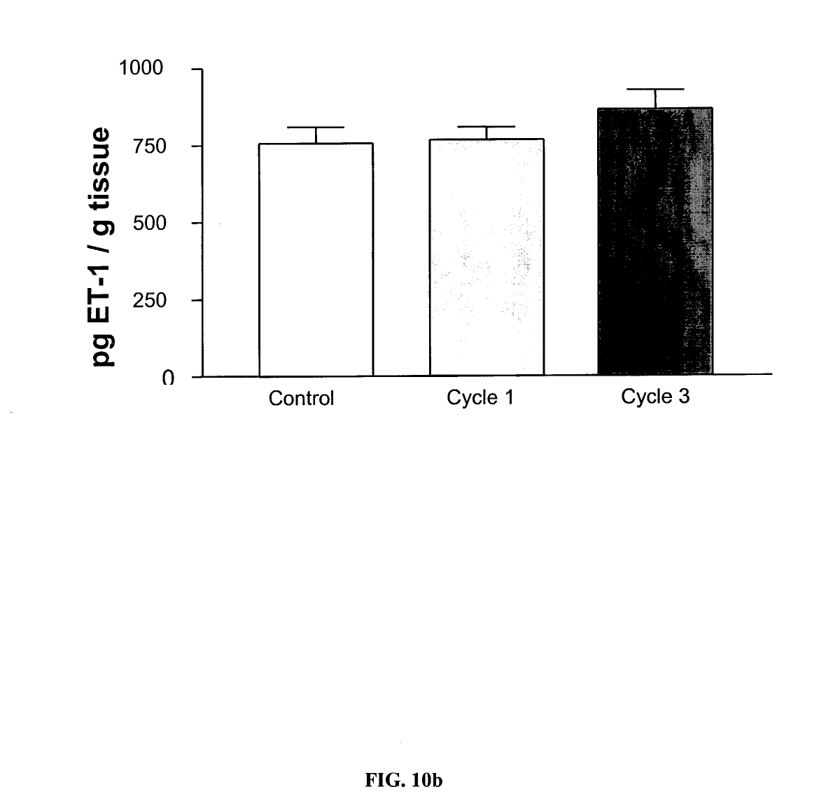

FIG. 10a illustrates that endothelin levels in the renal medulla were significantly increased during the third round of treatment with enalapril as compared to either control or cycle 1 (Con: 797±101.7, 1^{st} 767±8.0, 3^{rd} 1097±204.1 pg/g tissue). FIG. 10b shows that, in contrast, there was no change in endothelin levels in the cortex (Con: 756±118.4, 1^{st} 767±81.0, 3^{rd} 864±138 pg/g tissue). These data demonstrate that the progressive decrease in the magnitude of the persistent reduction in mean arterial pressure (MAP) following brief, repetitive antihypertensive therapy is due to increased renal medullary endothelin levels during the 3^{rd} treatment cycle.

10. Discussion

The major findings of these studies were that: (i) persistent lowering of mean arterial pressure (MAP) occurred after only two weeks of treatment with both an ACE inhibitor and an AT₁ receptor antagonist; (ii) there was a diminishing effect of repetitive drug challenges on the persistent lowering of MAP with both agents; and (iii) structurally-based vascular resistance properties were reduced in the first 2 weeks of treatment. These findings demonstrate that drug-induced changes occurring in the early phase of treatment contribute most importantly to the persistent lowering of pressure with longer treatments.

The present data demonstrate that the persistent lowering of MAP occurred regardless of whether the treatment involved an ACE inhibitor or AT₁ receptor antagonist. Surprisingly, regardless of the method of renin angiotensin system (RAS) inhibition, the first 2-week cycle of treatments that produced only a ˜28% decrease in MAP induced a ˜12% reduction in the off-treatment level of arterial pressure, i.e. ˜45% of the on-treatment depressor response persisted into the off-treatment period.

The second cycle of treatment induced a further 3-5% decrease in MAP off-treatment, indicating that maximal effects were not achieved after 2 weeks of RAS inhibition. However, given that the magnitude of the persistent change produced by the second treatment cycles is more than 60% reduced compared to the first cycle, it appears that the critical time for inducing persistent changes is during the early phase of treatment. This understanding is emphasized in the finding that the third treatment cycle did not produce any additional persistent effects on MAP. These observations suggest that the impact of the repetitive treatments on mechanisms that produce the persistent lowering of pressure becomes progressively less effective.

The mechanisms responsible for the RAS inhibitor-induced persistent lowering of pressure have not been fully elucidated, although structural changes in the vasculature (decreased media to lumen ratio) have been implicated. 2 weeks of ACE inhibition induces persistent changes in structurally-based vascular resistance. The present study shows that these vascular changes (decreased media bulk) are also evident following treatment with losartan. The degree of change in vascular resistance was similar to the degree of the persistent lowering of MAP. This data is supportive of a causal relationship between changes in vascular structure and blood pressure.

These results provide considerable insight into further characterization of the RAS inhibitor-induced persistent lowering response. This study indicates that a 2-week treatment with either an ACE inhibitor or an AT₁ antagonist can induce a persistent lowering of pressure in adult SHR with fully established hypertension. These results also indicate that the early phase of treatment is critical in inducing the changes that result in the persistent lowering of pressure with both RAS inhibitors. Further, the novel observations of this study regarding the diminishing effects of progressive treatments on the persistent lowering of pressure raises the possibility that tolerance to the critical drug-induced effects can occur with therapy.

Tolerance is defined as a decreased response to a drug after repeated administrations such that a higher dose is required to produce the same effect that was once observed with the lower dose. Consistent with this concept, in the present study, on-treatment MAP was decreased to a similar level during each treatment cycle, yet the off-treatment persistent lowering of pressure response was progressively diminished with each further treatment cycle. Cross tolerance occurs when repeated use of drugs in a given category confers tolerance not only to the drug being used but also to other drugs in the same structural and mechanistic category. The preliminary crossover experiment involving administration of crossover treatments successively (continuing with the 2-week on-off treatment pattern) instead of after a prolonged drug-free period demonstrated cross-tolerance by revealing that the blunted persistent lowering response induced by repetitive treatment with an ACE inhibitor and an AT₁ antagonist could not be reversed by administration of the alternate drug under these circumstances. Together, these findings indicate that repetitive RAS inhibitor treatment results in tolerance to the drug-induced on-treatment effects resulting in the off-treatment persistent lowering of pressure.

A major finding of the data presented above demonstrates that the progressive decrease in the magnitude of the persistent lowering of MAP following repetitive cycles of treatment correlated with an increase in renal medullary endothelin levels. Endothelin is a powerful vasoconstrictor, and elevated levels of endothelin are associated with a variety of pathological conditions exhibiting an up-regulation of structurally-based vascular resistance. Taken together, these results indicate that the rise in endothelin levels during subsequent cycles of treatment with an anti-pressor agent is mechanistically linked to the development of tolerance to the persistent MAP lowering effects and the induction of regression in vascular structure of the anti-pressor agent.

### EXAMPLE

A male patient complaining about fatigue, loss of libido, weight gain, and erectile dysfunction is first evaluated by the ADAM (Androgen Deficiency in Aging Male) questionnaire (Morley et al., “Validation of a screening questionnaire for androgen deficiency in aging males.” Metabolism, 2000 September;49(9):1239-42.) and IIEF (Rosen et al., “The International Index of Erectile Function (IIEF): a state-of-the-science review.” Int. J. Impot. Res., 2002 August;14(4):226-44). If positive with both ADAM and IIEF, he will then be tested for serum total testosterone concentration or serum bioavailable testosterone concentration. If the serum total testosterone concentration or bioavailable testosterone concentration is at either low or below the normal physiologic total testosterone (300-1100 ng/dl) or below the normal bioavailable testosterone (<70 ng/dl) concentration using the standard assays, he will be placed on androgen replacement therapy in order to bring the total testosterone into the middle of the normal range. He will be evaluated 2 or 3 months after initiation of the androgen therapy for his erectile function. If he is still considered as having erectile dysfunction based on IIEF, he will be given anti-pressor therapy while continuing the androgen therapy. Following the use of the combination of androgen replacement therapy and an anti-pressor agent, e.g. ACE inhibitor, he will resume his normal erectile function following one or two cycles of anti-pressor therapy. A preferred testosterone formulation is a topical composition having a pH value of between about 4 to about 8 and comprises: a) the hormone in a concentration of about 0.1% to about 2%, w/w (weight to weight) and b) a penetration-enhancing system consisting essentially of (i) a membrane fluidizer comprising oleic acid; (ii) a C₁-C₄ alcohol; (iii) a glycol, and (iv) optionally a gelling agent.

While there have been shown and described what are believed at present to be the preferred embodiments of the present invention, it will be obvious to those of ordinary skill in the art that various modifications can be made in the preferred embodiments without departing from the scope of the invention as it is defined by the appended claims.

What is claimed is:

1. A method for treating a vascular condition, said method comprising:
administering to a human patient in need thereof a therapeutically effective amount of a combination of at least two agents selected from the group consisting of an anti-pressor agent, an endothelin antagonist, and a sex hormone, wherein said vascular condition is treated.

2. The method of claim 1, wherein said vascular condition is selected from the group consisting of a sexual dysfunction, atherosclerosis, renal failure, hypertension, congestive heart failure, diabetic nephropathy, and diabetic neuropathy.

3. The method of claim 1, wherein said anti-pressor agent is selected from the group consisting of prostaglandin-E₁, an ACE inhibitor, an angiotensin-II receptor antagonist, an α₁-adrenergic receptor antagonist, a β-adrenergic receptor antagonist, a calcium channel blocker, an activator of guanylyl cyclase, an activator of adenyl cyclase, a phosphodiesterase inhibitor, and hydralazine.

4. The method of claim 3, wherein said ACE inhibitor is selected from the group consisting of alacepril, benazepril, captopril, ceronapril, cilazapril, delapril, enalapril, fosinopril, imidapril, lacidipine, libenzapril, lisinopril, moexipril, moveltipril, pentopril, perindopril, quinapril, ramipril, spirapril, temocapril, and trandolapril.

5. The method of claim 3, wherein said ACE inhibitor is enalapril.

6. The method of claim 3, wherein said angiotensin-II receptor antagonist is selected from the group consisting of eprosartan, irbesartan, losartan, and valsartan.

7. The method of claim 3, wherein said angiotensin-II receptor antagonist is losartan.

8. The method of claim 3, wherein said α₁-adrenergic receptor antagonist is selected from the group consisting of alfuzosin, apraclonidine, bunazosin, carvedilol, clonidine, dapiprazole, doxazosin, indoramin, labetolol, midrodrine, naphazoline, phenoxybenzamine, phentolamine, prazosin, tamsulosin, terazosin, trimazosin, and urapidil.

9. The method of claim 3, wherein said calcium channel blocker is selected from the group consisting of bepridil, diltiazem, mibrefadil, nicardipine, nifedipine, nimopidine, and verapamil.

10. The method of claim 3, wherein said activator of guanylyl cyclase or adenyl cyclase is selected from the group consisting of YC-1 and forskolin.

11. The method of claim 3, wherein said phosphodiesterase inhibitor is selected from the group consisting of amrinone and sildenafil.

12. The method of claim 1, wherein said endothelin antagonist is selected from the group consisting of a peptidal endothelin antagonist, a non-peptidal endothelin antagonist, and an inhibitor of endothelin converting enzyme.

13. The method of claim 12, wherein said peptidal endothelin antagonist is an ETA/ETB receptor antagonist.

14. The method of claim 13, wherein said ETA/ETB receptor antagonist is PD145065.

15. The method of claim 12, wherein said non-peptidal endothelin antagonist is bosentan.

16. The method of claim 12, wherein said inhibitor of endothelin converting enzyme is phosphoramidon.

17. The method of claim 1, wherein said sex hormone is a testosterone-like compound.

18. The method of claim 1, wherein said sex hormone is an estrogen-like compound.

19. The method of claim 17, wherein said at least two agents are an ACE inhibitor and testosterone.

20. The method of claim 1, wherein said endothelin antagonist eliminates or reduces anti-pressor tolerance.

21. The method of claim 1, wherein said at least two agents are co-administered for at least two treatment cycles separated by a drug-free period.

22. The method of claim 21, wherein said at least two treatment cycles have different durations.

23. The method of claim 21, wherein said at least two agents are co-administered for at least two treatment cycles of at least 7 days, with each said treatment cycle being separated by a drug-free period of at least 7 days.

24. The method of claim 21, wherein said at least two agents are co-administered for at least two treatment cycles of about 14 days, with each said treatment cycle being separated by a drug-free period of about 14 days.

25. The method of claim 21, wherein said at least two agents are co-administered for at least three treatment cycles separated by a drug-free period having different durations.

26. A method for treating a vascular condition associated with a male or female sexual dysfunction, said method comprising:
administering to a human patient in need thereof a therapeutically effective amount of a combination of at least two agents selected from the group consisting of an anti-pressor agent, an endothelin antagonist, and a sex hormone, wherein said vascular condition associated with a male or female sexual dysfunction is treated.

27. The method of claim 26, wherein said male sexual dysfunction is selected from the group consisting of erectile dysfunction, priapism, and premature ejaculation.

28. The method of claim 26, wherein said female sexual dysfunction is selected from the group consisting of vaginal lubrication, vaginal engorgement, pain during intercourse, dyspareunia, an urogenital infection, post-menopause, diabetes, vascular disease, an estrogen depletion condition, idiosyncratic vaginal dryness, vaginismus, vulvodynia, interstitial cystitis, nonspecific urethritis, a sexual arousal disorder, hypoactive desire disorder and a sexual orgasmic disorder.

29. The method of claim 26, wherein said anti-pressor agent is selected from the group consisting of prostaglandin-E1, an ACE inhibitor, an angiotensin-II receptor antagonist, an α₁-adrenergic receptor antagonist, a β-adrenergic receptor antagonist, a calcium channel blocker, an activator of guanylyl cyclase, an activator of adenyl cyclase, a phosphodiesterase inhibitor, and hydralazine.

30. The method of claim 29, wherein said ACE inhibitor is selected from the group consisting of alacepril, benazepril, captopril, ceronapril, cilazapril, delapril, enalapril, fosinopril, imidapril, lacidipine, libenzapril, lisinopril, moexipril, moveltipril, pentopril, perindopril, quinapril, ramipril, spirapril, temocapril, and trandolapril.

31. The method of claim 29, wherein said ACE inhibitor is enalapril.

32. The method of claim 29, wherein said angiotensin-II receptor antagonist is selected from the group consisting of eprosartan, irbesartan, losartan, and valsartan.

33. The method of claim 29, wherein said angiotensin-II receptor antagonist is losartan.

34. The method of claim 29, wherein said α₁-adrenergic receptor antagonist is selected from the group consisting of alfuzosin, apraclonidine, bunazosin, carvedilol, clonidine, dapiprazole, doxazosin, indoramin, labetolol, midrodrine, naphazoline, phenoxybenzamine, phentolamine, prazosin, tamsulosin, terazosin, trimazosin, and urapidil.

35. The method of claim 29, wherein said calcium channel blocker is selected from the group consisting of bepridil, diltiazem, mibrefadil, nicardipine, nifedipine, nimopidine, and verapamil.

36. The method of claim 29, wherein said activator of guanylyl cyclase or adenyl cyclase is selected from the group consisting of YC-1 and forskolin.

37. The method of claim 29, wherein said phosphodiesterase inhibitor is selected from the group consisting of amrinone and sildenafil.

38. The method of claim 26, wherein said endothelin antagonist is selected from the group consisting of a peptidal endothelin antagonist, a non-peptidal endothelin antagonist, and an inhibitor of endothelin converting enzyme.

39. The method of claim 38, wherein said peptidal endothelin antagonist is an ET_{A}/ET_{B} receptor antagonist.

40. The method of claim 39, wherein said ETA/ETB receptor antagonist is PD145065.

41. The method of claim 38, wherein said non-peptidal endothelin antagonist is bosentan.

42. The method of claim 38, wherein said inhibitor of endothelin converting enzyme is phosphoramidon.

43. The method of claim 26, wherein said sex hormone is a testosterone-like compound.

44. The method of claim 26, wherein said sex hormone is an estrogen-like compound.

45. The method of claim 43, wherein said at least two agents are an ACE inhibitor and testosterone.

46. The method of claim 26, wherein said endothelin antagonist eliminates or reduces anti-pressor tolerance.

47. The method of claim 26, wherein said at least two agents are co-administered for at least two treatment cycles separated by a drug-free period.
